(19) United States
(12) Patent Application Publication
(10) Pub. No.: US 2009/0108733 A1
(43) Pub. Date: Apr. 30, 2009

(54) Precursors of Organometallic Compounds for Electroluminescent Materials

(75) Inventors: Sung-Min Chin, Chungcheong-bukdo (KR); Sang-Man Si, Seoul (KR); Keun-Hee Han, Kyeongki-do (KR); Kyung-Hoon Choi, Seoul (KR); Mi-Young Kwak, Seoul (KR); Bong-Ok Kim, Seoul (KR); Sung-Min Kim, Seoul (KR)

Correspondence Address: CLARK & BRODY, 1090 VERMONT AVENUE, NW, SUITE 250, WASHINGTON, DC, 20005, US

(73) Assignee: GRACEL DISPLAY INC., Seoul (KR)

(21) Appl. No.: 11/922,675

(22) PCT Filed: Jan. 22, 2006
(86) PCT No.: PCT/KR2006/000243

§ 371 Date: Dec. 21, 2007

(30) Foreign Application Priority Data
Jun. 23, 2005 (KR) 10-2005-0054228

(51) Int. Cl. H01L 51/54; C07D 277/66; C07D 513/04; C07F 3/06

(52) U.S. Cl. 313/504; 548/156; 548/106; 548/151; 548/108

(57) ABSTRACT

The present invention relates to a precursor of metallic complex for electroluminescent material, a luminescent material comprised of said precursor of metallic complex and metal, and an electroluminescent device containing a luminescent material comprised of said precursor and metal. The luminescent material comprised of the precursor according to the present invention and metal is advantageous in that it shows excellent electric conductivity and light emitting property of high efficiency.

### TECHNICAL FIELD

The present invention relates to a precursor of metallic complex for electroluminescent material, a luminescent material comprised of said precursor of metallic complex and metal, and an electroluminescent device containing a luminescent material comprised of said precursor and metal.

### BACKGROUND ART

Among display devices, organic electroluminescence (EL) devices, being self-luminous type display devices, have advantages of wide visual angle, excellent contrast as well as rapid response rate.

Meanwhile, Eastman Kodak firstly developed an organic EL device employing low molecular aromatic diamine and aluminum complex as a substance for forming a light emitting layer, in 1987 [Appl. Phys. Lett. 51, 913, 1987].

Though fluorescent materials have been widely used up to the present as the light emitting material, the most important factor to determine luminous efficiency in an organic EL device, development of phosphor material, from the aspect of the mechanism of electroluminescence, is one of the best ways to improve the luminous efficiency up to 4 folds, theoretically.

Up to the present, iridium (III) complexes have been widely known as phosphorescent light emitting material: (acac)Ir(btp)₂, Ir(ppy)₃ and Firpic or the like having been known as RGB, respectively. More improved phosphorescent materials are expected to be reported, because various phosphors have been recently researched in Korea, Japan, Europe and America, in particular.

As a host material for phosphorescent light emitting material, 4,4′-N,N′-dicarbazole-biphenyl (CBP) is most widely known up to the present, and a hole blocking layer has been applied with said CBP to develop OLEDs having high efficiency. Further, East-North Pioneer (Japan) or the like has developed OLEDs of high performance by using bis(2-methyl-8-quinolinato)(p-phenylphenolato)aluminum (III) (BAlq) and its derivatives as the host for phosphorescent light emitting material.

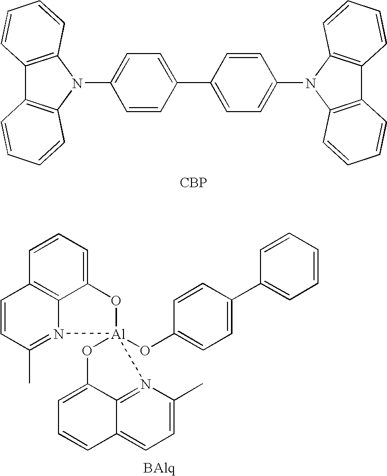

In the meanwhile, a hetero atom contained in an aromatic ring or containing unshared electron pair as a side chain substituent has a property to readily form a coordination bond to metal. Such a coordination bond shows very stable property from electrochemical aspect, which has been the widely known property of complexes. By applying the property of said complexes, researches on metal complexes for light emitting material have been performed since the middle of 1990's, such as metal complexes represented by Chemical Formula A [2-(2-hydroxyphenyl)benzoxazoles: Appl. Phys. Lett., 64, 815, 1994] and azomethine metal complexes represented by Chemical Formula B [Jpn. J. Appln. Phys., 32, 1511, 1993] adopted in blue light emitting materials.

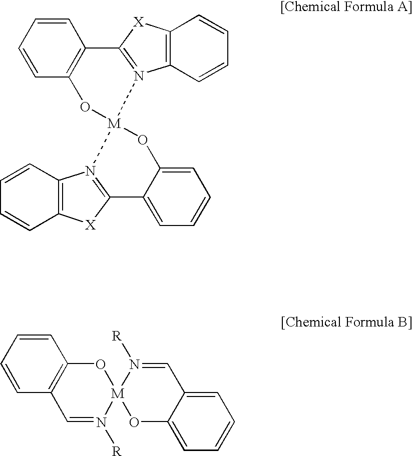

However, the metal complexes up to the present have restriction in terms of electric conductivity and luminous efficiency.

### DISCLOSURE

### Technical Problem

The object of the present invention is to solve the problems described above and to provide precursors of metal complex for luminescent material to give excellent metal complexes exhibiting far better electric conductivity and light emitting properties of high efficiency as compared to conventional materials. Another object of the invention is to provide a luminescent material comprised of the precursor of metal complex as prepared and metal, and an electroluminescent device comprising the same.

### Technical Solution

The present invention relates to a precursor of metallic complex for electroluminescent material represented by Chemical Formula 1, a luminescent material comprised of said precursor of metallic complex and metal, and an electroluminescent device containing a luminescent material comprised of said precursor and metal.

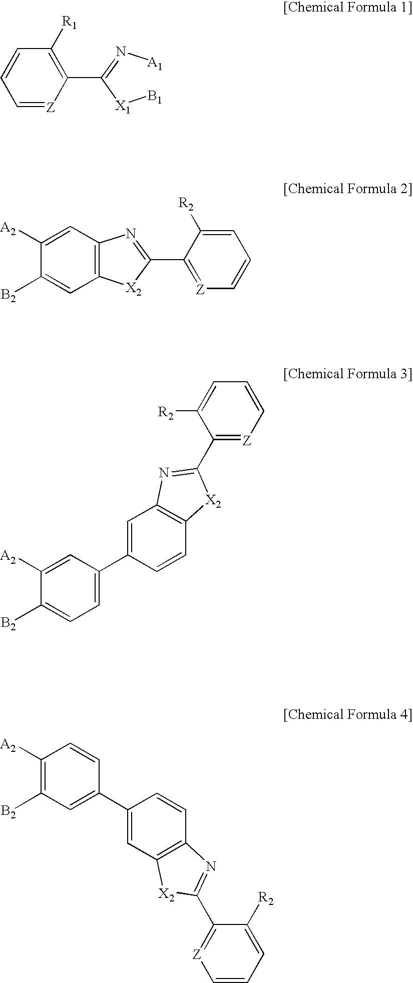

A compound represented by Chemical Formula 1 forms or A₁-B₂/B₁-A₂ single bond with each substituent selected from the groups represented by one of Chemical Formulas 2 to 4; if Z is carbon (C), each X₁ and X₂ independently represents O, S, Se or N-Ph, and each R₁ and R₂ independently represents NH₂, OH or SH; if Z is nitrogen (N), each X₁ and X₂ independently represents NH or PH, and each R₁ and R₂ is hydrogen.

The compounds represented by Chemical Formula 1 include precursors of metal complexes for luminescent material represented by one of Chemical Formulas 5 to 8.

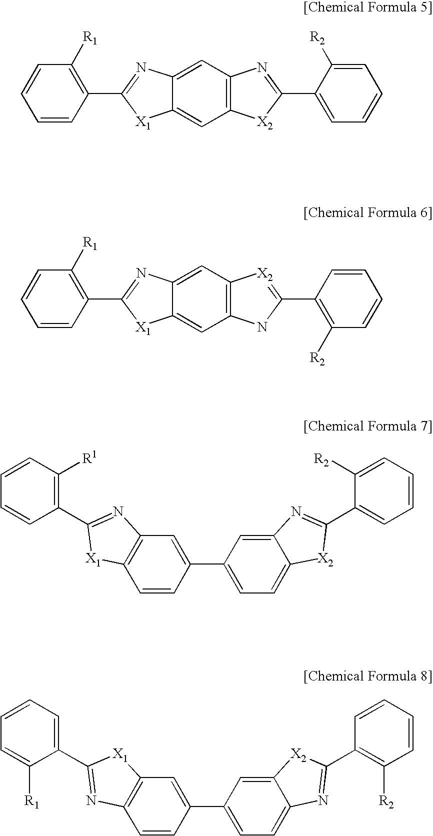

In Chemical Formulas 5 to 8, each X₁ and X₂ independently represents O, S, Se or N-Ph, and each R₁ and R₂ independently represents NH₂, OH or SH.

The compounds represented by Chemical Formula 1 also include precursors of metal complexes for luminescent material represented by one of Chemical Formulas 9 to 12.

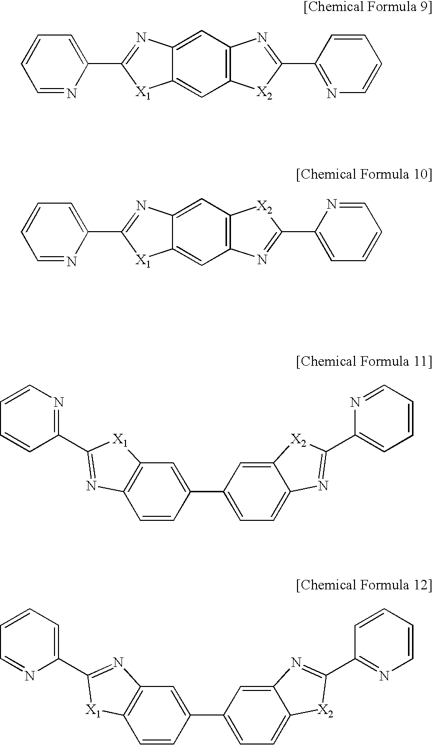

In Chemical Formulas 9 to 12, each X₁ and X₂ independently represents NH or PH.

Specifically, the precursors of metallic complexes for luminescent material represented by one of Chemical Formulas 5 to 12 may be exemplified as follows:

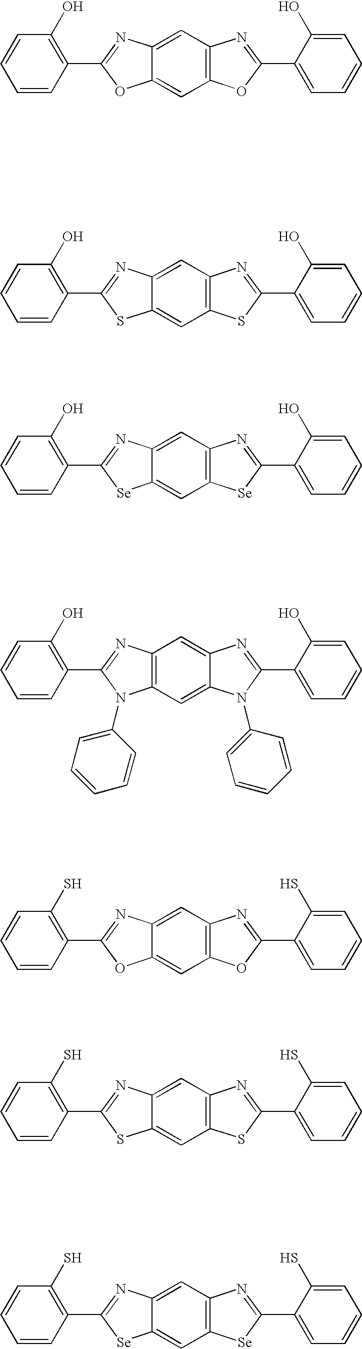

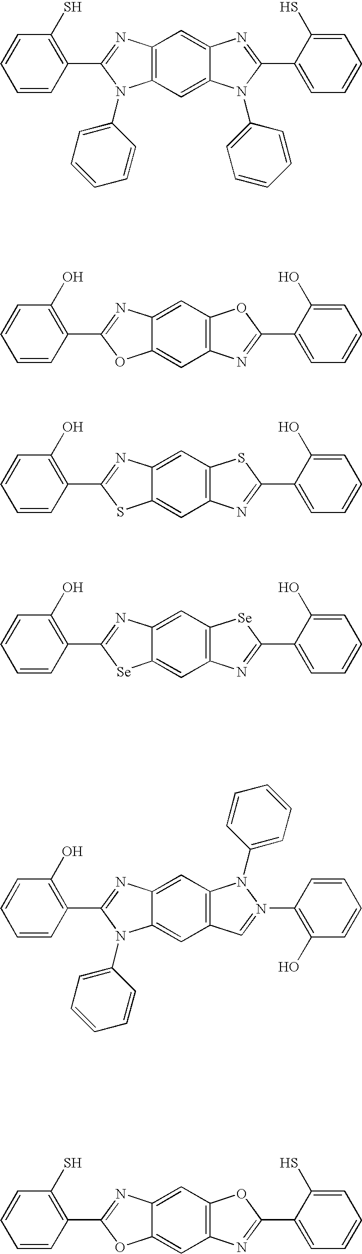

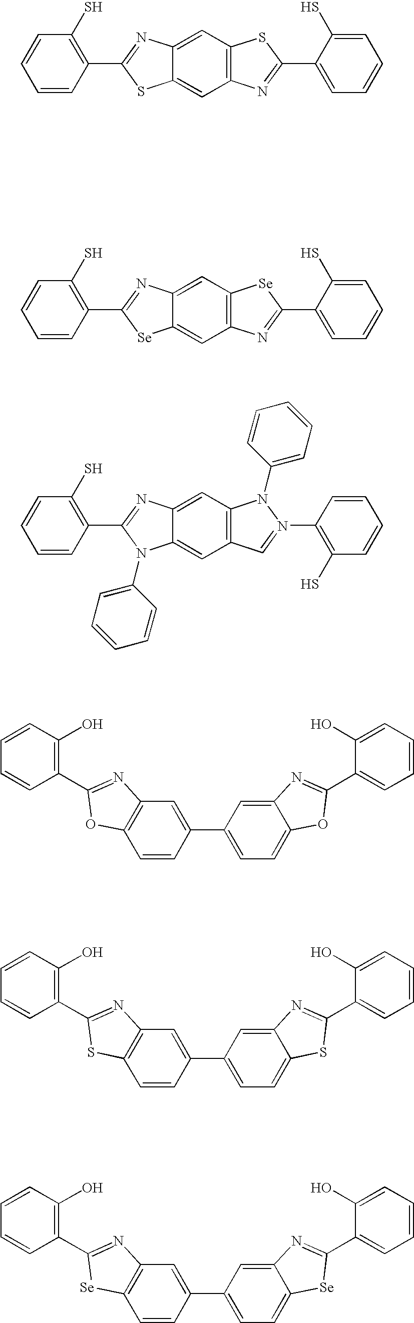

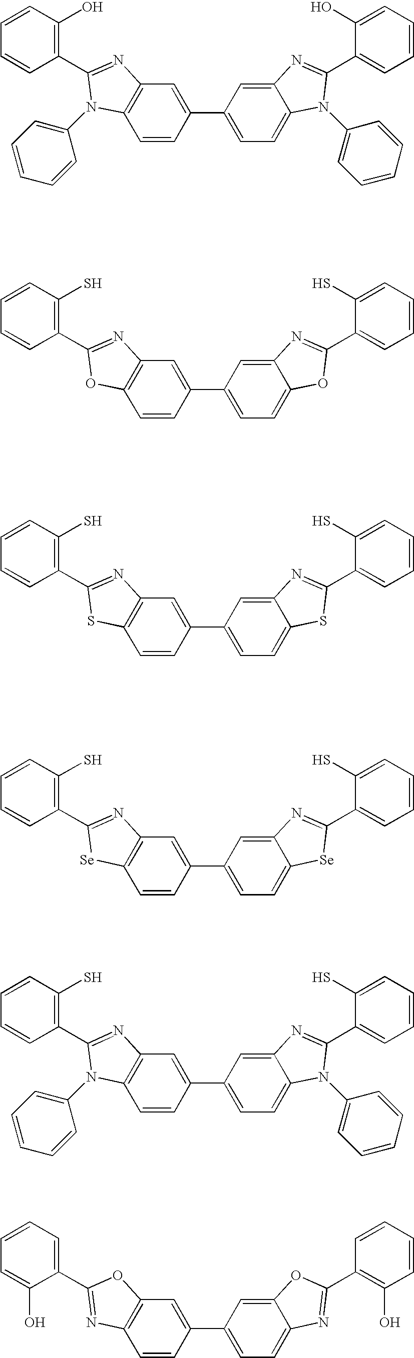

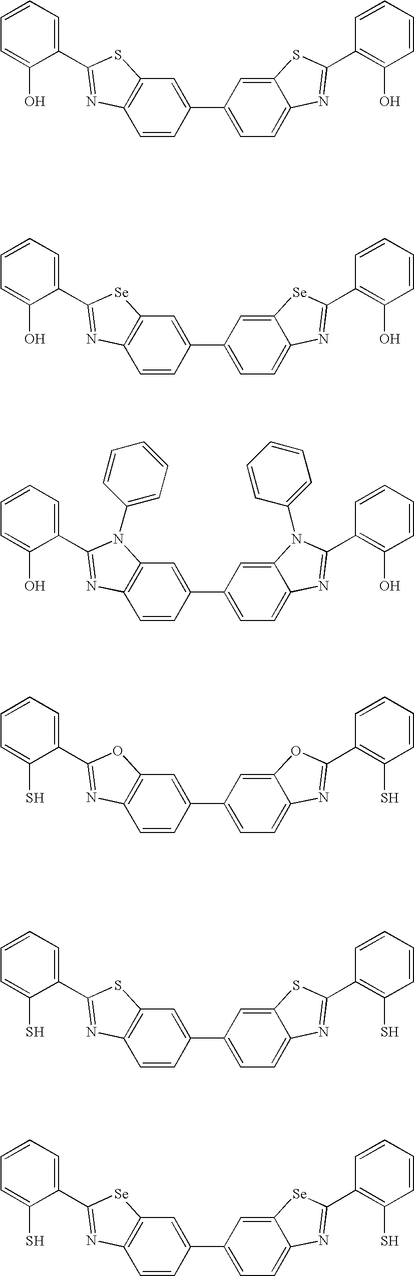

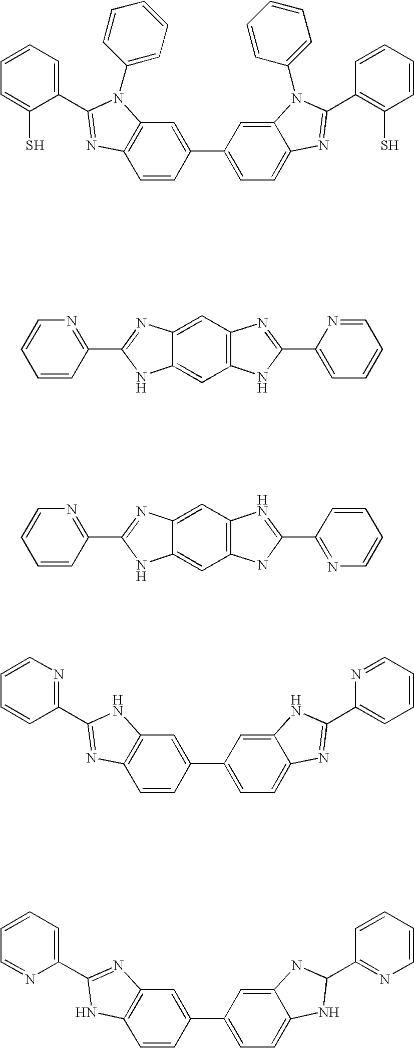

Compounds represented by one of Chemical Formulas 5 and 9 as the precursor of metallic complexes for luminescent material can be prepared via the reaction route illustrated by Reaction Scheme 1, while compounds represented by one of Chemical Formulas 6 and 10 can be prepared via the reaction route illustrated by Reaction Scheme 2.

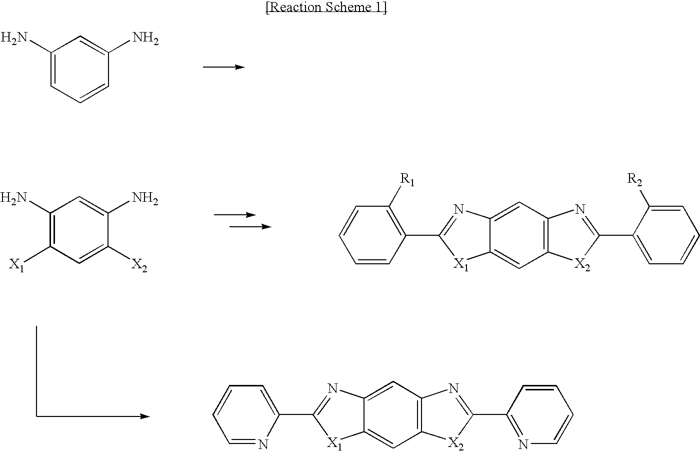

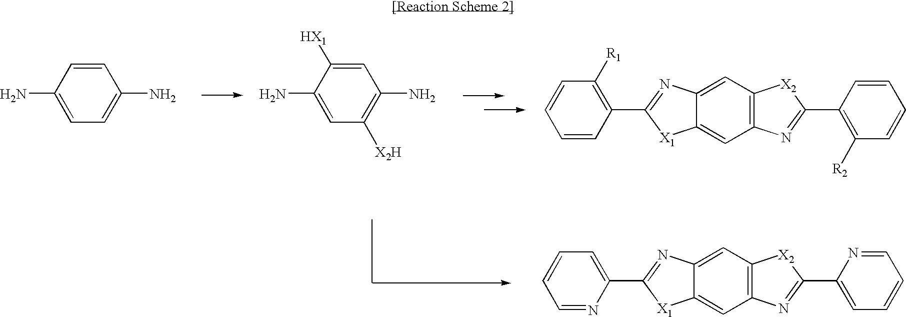

The biphenyl derivatives represented by one of Chemical Formulas 8 to 11 can be prepared via same reaction route as illustrated by Reaction Scheme 3, being similar to Reaction Scheme 1 and Reaction Scheme 2, and the compounds represented by one of Chemical Formulas 7 to 12 can be prepared by a similar method to one of Reaction Schemes 1 to 3.

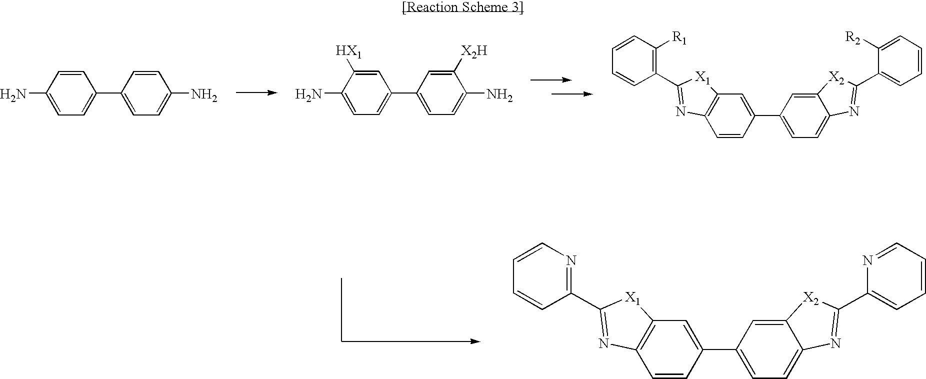

A precursor of metal complex according to the present invention reacts with metal salt such as Be, Zn, Mg or Al to form a metal complex for luminescent material. The metal complex according to the invention forms a complex by coordination with a metal ion between the precursor compound molecules depending on the structure of the precursor compound as illustrated by one of Chemical Formula 13 and 14 with a total composition ratio of the precursor to metal ion being 1:1. The reaction condition is described as follows:

A precursor compound (1.0 mmol) is charged in 30-50 mL of methanol, and 2.2 mmol of sodium hydroxide is added thereto, and the resultant mixture is stirred vigorously to form a solution. To the solution, a solution of metal salt such as beryllium sulfate (II), zinc acetate (II) or magnesium (II) acetate dissolved in 5 mL of methanol is slowly added, and the mixture stirred at ambient temperature for 2 hours. The resultant precipitate is filtered and washed with 20 mL of distilled water, 50 mL of methanol and 10 mL of ethyl ether, and then dried under vacuum.

The yield of complex of the precursor compound and metal according to the present invention is 30-50%, 50˜70%, or 10˜40%, depending on the metal salt employed, that is beryllium (II) sulfate, zinc (II) acetate, magnesium (II) acetate, respectively. The yield may vary depending upon the structure of the precursor.

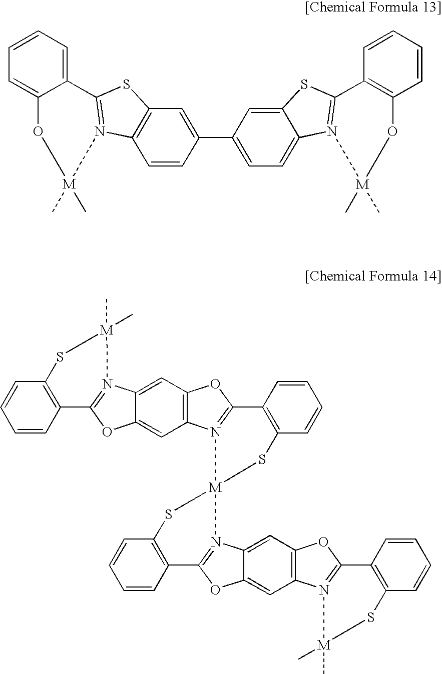

The metal complex for luminescent material comprising the precursor compound and metal complex according to the present invention is vapor deposited with other luminescent material as a light emitting layer of electroluminescent device, by means of vapor deposition device.

### DESCRIPTION OF DRAWINGS

FIG. 1 is an EL spectrum of an OLED device prepared according to Comparative Example 1;

FIG. 2 shows current density-voltage property of the OLED device prepared according to Comparative Example 1;

FIG. 3 shows luminous efficiency-luminance property of the OLED device prepared according to Comparative Example 1;

FIG. 4 shows current density-voltage property of an OLED device prepared according to Example 1;

FIG. 5 shows luminous efficiency-luminance property of the OLED device prepared according to Example 1;

FIG. 6 shows current density-voltage property of an OLED device prepared according to Example 2;

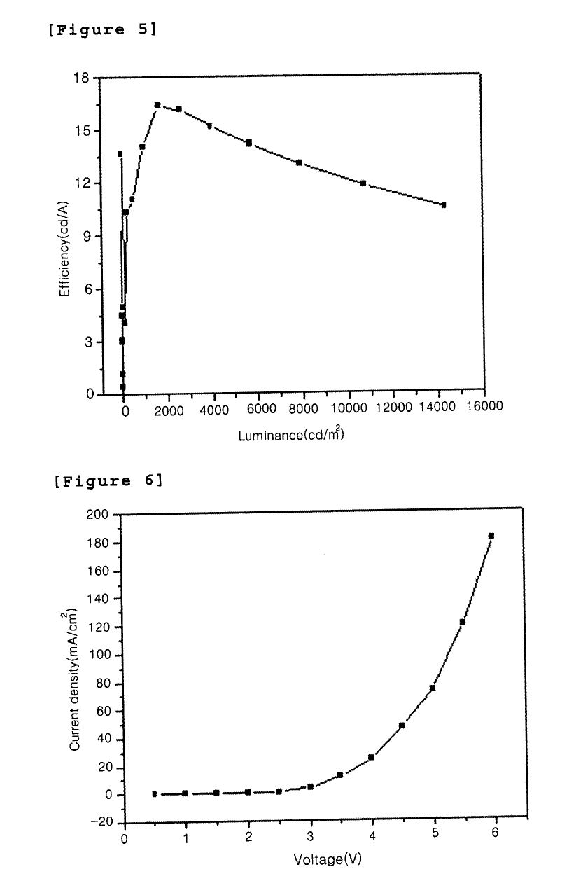

FIG. 5 shows luminous efficiency-luminance property of the OLED device prepared according to Example 2.

### MODE FOR INVENTION

Now, the present invention is described as referring to exemplary processes for preparing novel precursor compounds for luminescent material and metal complexes of said precursor with metal ion according to the present invention, as well as processes for preparing electroluminescent devices employing the luminescent material of metal complex thus prepared and light emitting properties of said the luminescent device by way of Examples. These Examples, however, are intended to provide better understanding of the invention, and it should be understood that the scope of the invention is not restricted thereto.

### Synthetic Example 1

### Synthesis of DPBT

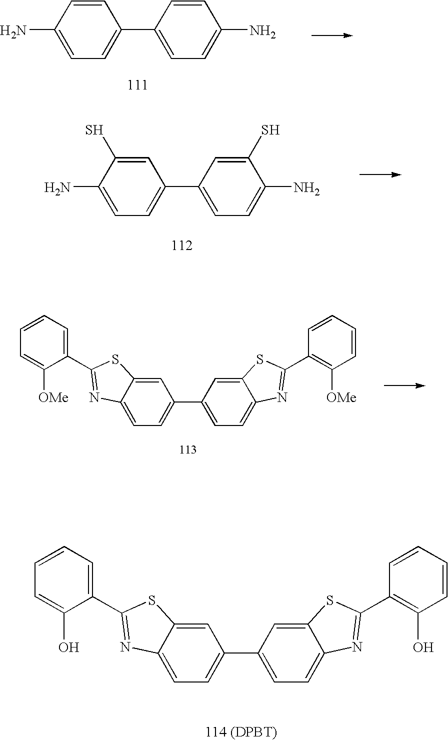

Benzidine (1.0 g, 5.4 mmol) and potassium thiocyanate (2.4 g, 24.4 mmol) were charged to 20 mL of acetic acid, and the resultant mixture was stirred at ambient temperature for 10 minutes. After slowly adding 0.5 mL (1 eq) of bromine thereto, the resultant mixture was stirred at ambient temperature for 2 hours. Yellow precipitation appears after 1 hour lapse of stirring. After completion of stirring, 20 mL of methanol was added to the reaction solution, and the resultant mixture was neutralized with 0.1N aqueous potassium hydroxide solution. To the neutralized reaction solution, 40˜50 mL of distilled water is added to obtain solid thus formed, which was then filtered and washed with distilled water and methanol to give 1.2 g (4:1 mmol, yield: 75%) of intermediate. The intermediate was charged in 15 mL of 2,3-butandiol, and the mixture was warmed to form a solution. An excess amount of potassium hydroxide was added thereto, and the mixture was heated to 200° C. under reflux for 3 hours. The mixture was neutralized with appropriate amount of acetic acid with lowering the temperature. When reaching ambient temperature, the mixture was extracted with ethyl ether to obtain compound (112) (0.75 g, 3.0 mmol, yield: 73%).

After dissolving the compound (112) thus obtained 10 (0.75 g, 3.0 mmol) in 15 mL of DMSO, 2-methoxybenzaldehyde (0.8 mL, 6.6 mmol) was added and mixture was heated. When the temperature of the reaction solution reached 180° C. or more, the reaction was not raised any more, and maintained the temperature of the reaction mixture for one hour. The temperature was lowered to ambient temperature, and at least 20 mL of distilled water was added to form precipitate, which was then washed with excess amount of distilled water and n-hexane, and dried to obtain compound (113) (1.2 g, 2.5 mmol, yield 83%).

¹H NMR (200 MHz, CDCl₃): δ 3.8 (s, 6H), 6.78-6.9 (m, 4H), 7.1-7.14 (m, 2H), 7.35-7.4 (d, 2H), 7.75-7.8 (d, 2H), 8.25-8.35 (m, 4H)

MS: 480 (found), 480.61 (calculated)

To a solution of compound (113) prepared above (1.2 g, 2.5 mmol) dissolved in 30 mL of 1,2-dichloroethane, added was boron tribromide-dimethylsulfide complex (8.0 g, 5 eq. 25.6 mmol), and the mixture was stirred at 85° C. for hours. After cooling to ambient temperature, 0.1N aqueous HCl solution was added to quench the reaction. Extraction with methylene chloride provided DPBT (0.9 g, 2.0 mmol, yield: 80%), as the title compound.

¹H NMR (200 MHz, CDCl₃): δ 4.7 (s, 2H), 6.8-7.1 (m, 6H), 7.3 (d, 2H), 7.8 (d, 2H), 8.3-8.4 (m, 4H)

### Synthetic Example 2

### Synthesis of Zn-DPBT

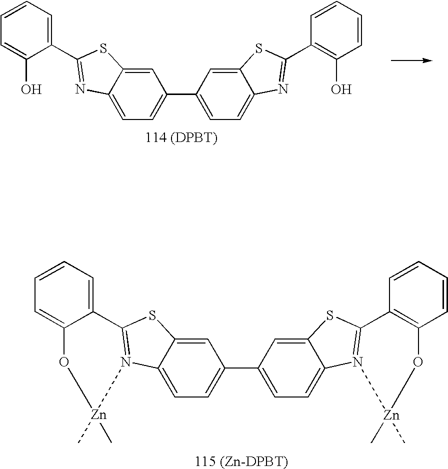

To 30 mL of methanol, charged was DPBT (0.9 g, 2.0 mmol) prepared in Synthetic Example 1, and sodium hydroxide (at least 0.5 eq) was added thereto. The mixture was stirred to form a solution. A solution of zinc (II) acetate (0.35 g, 2.2 mmol) dissolved in 5 mL of methanol was slowly added to the reaction mixture, and the resultant mixture stirred at ambient temperature for 2 hours. The precipitate generated was filtered from the reaction solution, and washed with 20 mL of distilled water, 50 mL of methanol and 10 mL of ethyl ether, and dried under vacuum to obtain the title compound, Zn (II) complex of DPBT (Zn-DPBT) (0.9 g, yield: 74%).

MS/FAB: 516, 1032, 1546 (found)

### Synthetic Example 3

### Synthesis of DMBT

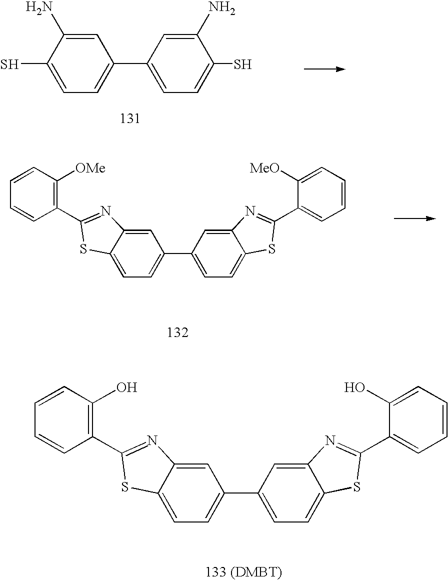

Same procedure as Synthetic Example 1 was repeated but using 3,3′-diaminobiphenyl-4,4′-dithiol (1.0 g, 4.0 mmol) as compound (112) to obtain the title compound (133) (DMBT) (0.75 g, 1.66 mmol, yield: 42%).

¹H NMR (200 MHz, CDCl₃): δ 4.7 (s, 2H), 6.8-7.1 (m, 6H), 7.3 (d, 2H), 7.8 (d, 2H), 8.2 (d, 2H), 8.4 (s, 2H)

### Synthetic Example 4

### Synthesis of Zn-DMBT

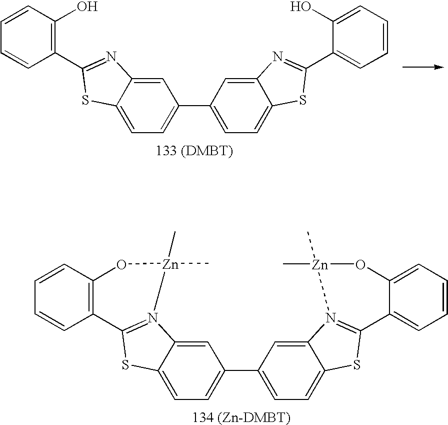

Same procedure as Synthetic Example 2 was repeated but using DMBT (0.75 g, 1.66 mmol) obtained as above instead of using DPBT to obtain the title compound, Zn (II) complex of DMBT (compound 134, Zn-DMBT) (0.5 g, yield: 58%).

MS/FAB: 516, 1032, 1546 (found)

### Synthetic Example 5

### Synthesis of 3-DPDB

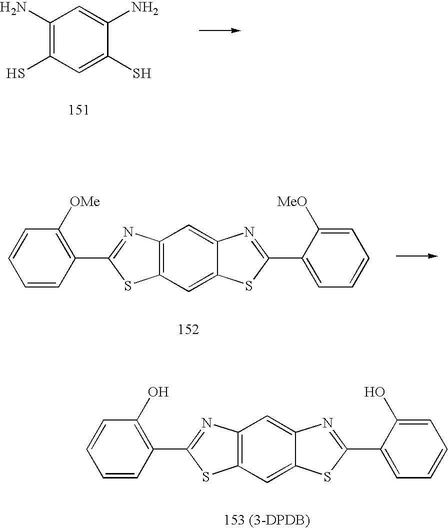

Same procedure was repeated as Synthetic Example 1 but using compound (151) (1.0 g, 5.8 mmol) as compound (112) to obtain the title compound (153, 3-DPDB) (0.65 g, 1.7 mmol, yield: 29%).

¹H NMR (200 MHz, CDCl₃): δ 4.8 (s, 2H), 6.8-7.1 (m, 6H), 7.3 (d, 2H), 8.1 (s, 1H), 8.3 (s, 1H)

### Synthetic Example 6

### Synthesis of Zn-3-DPDB

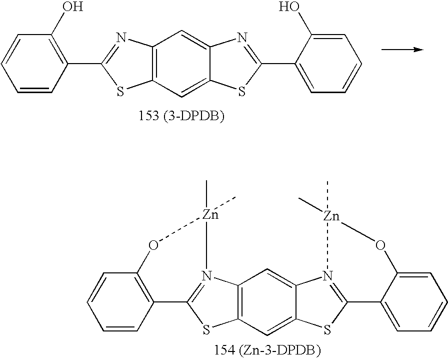

Same procedure as Synthetic Example 2 was repeated but using 3-DPDB (0.65 g, 1.7 mmol) obtained as above instead of using DPBT to obtain the title compound, Zn (II) complex of 3-DPDB (compound 154, Zn-3-DPDB) (0.3 g, yield: 40%).

MS/FAB: 439, 879 (found)

### Synthetic Example 7

### Synthesis of 4-DPDB

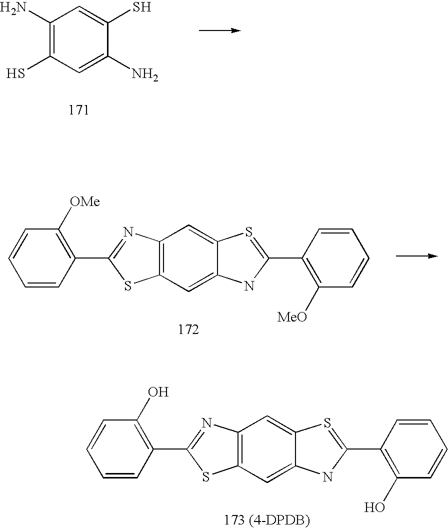

Same procedure was repeated as Synthetic Example 4 but using compound (171) (1.0 g, 5.8 mmol) as compound (112) to obtain the title compound (173, 4-DPDB) (0.8 g, 2.1 mmol, yield: 36%).

¹H NMR (200 MHz, CDCl₃): δ 4.85 (s, 2H), 6.75-7.1 (m, 6H), 7.3 (d, 2H), 8.15 (s, 2H)

### Synthetic Example 8

### Synthesis of Zn-4-DPDB

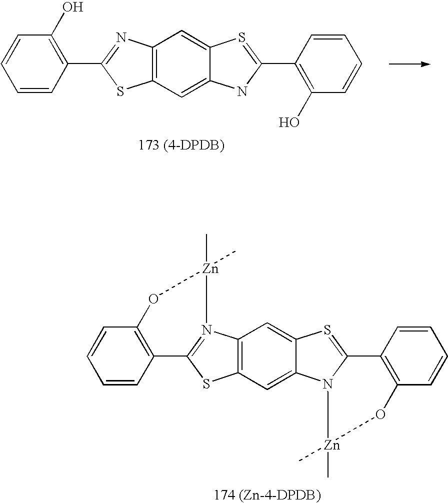

Same procedure as Synthetic Example 2 was repeated but using 4-DPDB (0.8 g, 2.1 mmol) obtained as above instead of using DPBT to obtain the title compound, Zn (II) complex of 4-DPDB (compound 174, Zn-4-DPDB) (0.25 g, yield: 27%).

MS/FAB: 439, 879, 1318 (found)

### Example 1

### Manufacture of OLED Device Having a Structure Employing the Luminescent Material of Metal Complex According to the Present Invention

A transparent electrode ITO thin film (15Ω/□) obtained from glass for OLED (manufactured from Samsung-Corning) was subjected to ultrasonic washing sequentially with trichloroethylene, acetone, ethanol and distilled water, and stored in isopropanol.

Then, an ITO substrate is equipped on a substrate folder of a vacuum vapor deposition device, and 4,4′,4″-tris(N,N-(2-naphthyl)-phenylamino)triphenylamine (2-TNATA) was charged in a cell of the vacuum vapor deposition device. After ventilation to reach the degree of vacuum in the chamber of 10⁻⁶ torr, electric current was applied to the cell to evaporate 2-TNATA to vapor-deposit a hole injecting layer on the ITO substrate with 60 nm of thickness.

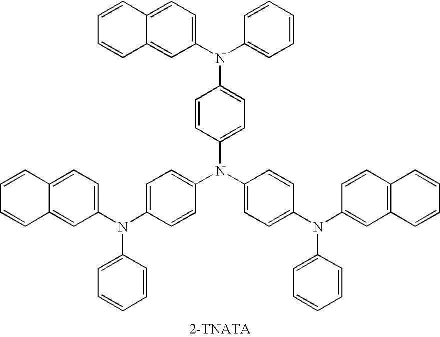

Then, N,N′-bis(α-naphthyl)-N,N′-diphenyl-4,4′-diamine (NPB) was charged in another cell of said vacuum vapor deposition device, and electric current was applied to the cell to evaporate NPB to vapor-deposit a hole transport layer with 20 nm of thickness on the hole injecting layer.

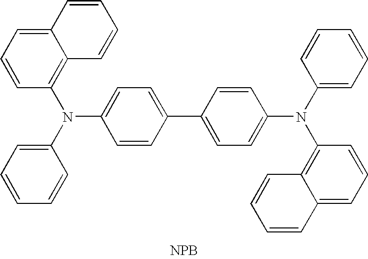

After forming the hole injecting layer and hole transport layer, metal complex Zn-DPBT according to the present invention (1:1 metal complex of DBPT prepared in Synthetic Example 1 and Zn(II) ion) was charged to another cell of the vapor deposition device, while (NPy)₂Ir(acac) represented by the Chemical Formular below as another luminescent material in still another cell. The two substances were doped by evaporating them in different rates, to vapor-deposit a light emitting layer having 30 nm of thickness with 4 to 10 mol % concentration on the hole transport layer.

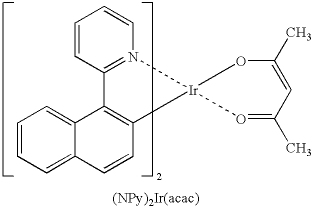

Then, tris(8-hydroxyquinoline)-aluminum(III) (Alq) was vapor-deposited with a thickness of 20 nm. Lithium quinolate (Liq) as an electron injecting layer was then vapor-deposited with a thickness of 1 to 2 nm, and Al cathode was vapor-deposited with a thickness of 150 nm by using another vapor deposition device, to manufacture an OLED.

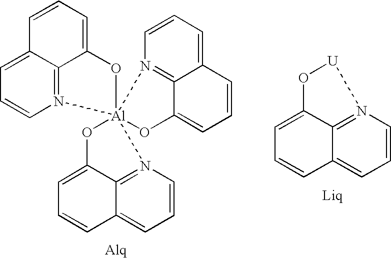

### Example 2

An OLED device was manufactured as described in Example 1, but Zn-DPBT as a metal complex (vapor-deposited in the light emitting layer in Example 1) according to the invention was vapor-deposited as an electron transport layer with a thickness of 20 nm.

### Comparative Example 1

An OLED device was manufactured as described in Example 1, but to another cell of the vapor deposition device, bis(2-methyl-8-quinolinato)(p-phenylphenolato)aluminum (III) (BAlq) was charged as a host luminescent material, while (NPy)₂Ir(acac) to still another cell, and the two substances were doped by evaporating them in different rates, to vapor-deposit a light emitting layer having 30 nm of thickness with 4 to 10 mol % of doping concentration on the hole transport layer.

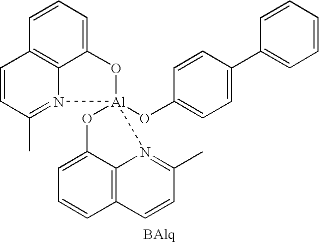

### Example 3

### Evaluation of Properties of OLED

Luminous efficiency was measured at 2,000 cd/m² and 10,000 cd/m² in order to evaluate the performance of each OLED prepared in Examples 1 and 2, and Comparative Example 1.

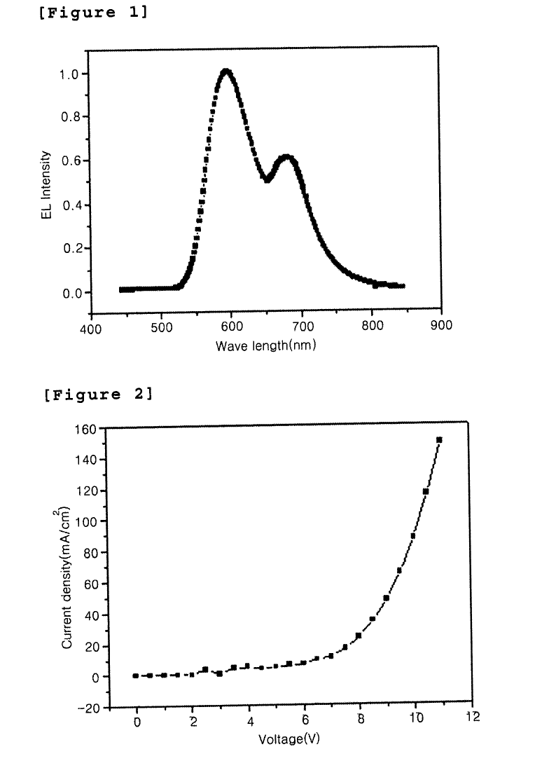

FIG. 1 is an EL spectrum of Comparative Example 1, which employs (NPy)₂Ir(acac) compound emitting orange-red light as luminescent material, and BAlq as a host, showing the maximum light emitting peak at about 597 nm.

When the metal complex according to the present invention is applied as a light emitting layer, 2-4 nm red shift of the EL spectrum could be observed, exhibiting rather advantageous effect from the aspect of color purity.

FIG. 2 shows current density-voltage property of Comparative Example 1. As can be shown in FIG. 2, the driving voltage of the device of Comparative Example 1 was about 6 V, and current density at 10 V was about 88 mA/cm².

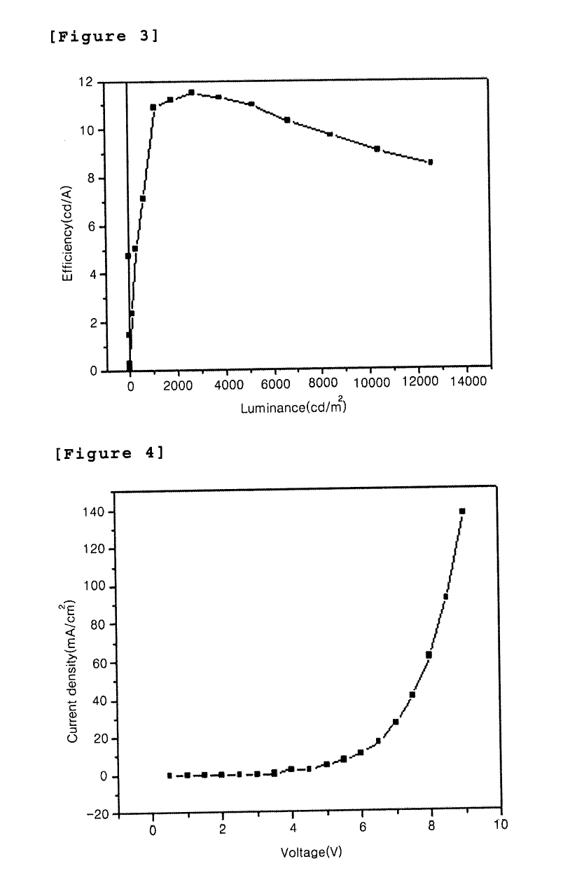

FIG. 3 shows luminous efficiency-luminance property of Comparative Example 1, wherein the luminous efficiency at about 2,000 cd/m² of luminance is about 11.3 cd/A, while about 9.2 cd/A of luminous efficiency at 10,000 cd/m² of luminance.

FIG. 4 shows current density-voltage property of the OLED device prepared according to Example 1. As can be seen, driving voltage of the device of Example 1 employing metal complex luminescent material comprised of precursor according to the present invention and metal was about 2.5 to 3 V, shows current density of about 88 mA/cm² at about 8.5 V, which means that the driving voltage is lower by about 1.5 V than that of OLED device of Comparative Example 1.

As can be seen from FIG. 5 showing the luminous efficiency-luminance property of the device of Example 1, luminous efficiency was 16.3 cd/A at 2,000 cd/m², and about 12.1 cd/A at 10,000 cd/m²: This is higher luminous efficiency at the same luminance by about 3-5 cd/A, as compared to the device prepared according to Comparative Example 1.

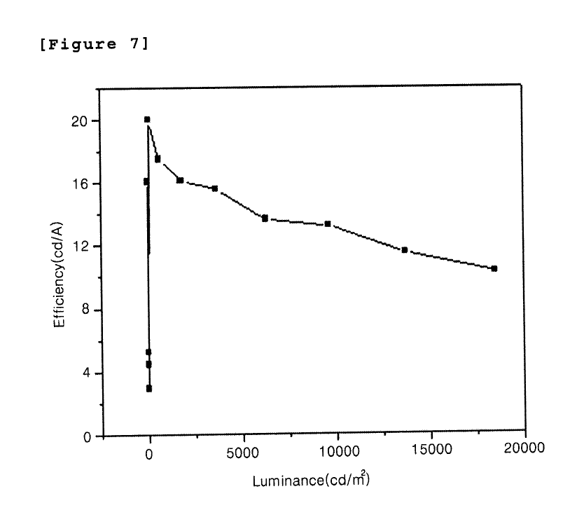

On the contrary, as can be seen from FIGS. 6 and 7 showing current density-voltage property and luminous efficiency-luminance of Example 2 where Zn-DPBT, metal complex according to the present invention was applied as light emitting layer and electron transport layer simultaneously, current density was about 88 mA/cm2 at about 5.2 V. This shows lowering of driving voltage by at least 4.5 V as compared to the device of Comparative Example 1, far lower than that of device of Example 1.

Further, the device according to Example 1 exhibited 16.1 cd/A of luminous efficiency at 2,000 cd/m² of luminance, and 13.0 cd/A at 10,000 cd/m²: this shows better luminous efficiency by about 4-5 cd/A as compared to the device of Comparative Example 1.

Power efficiency that is important in actual panel is in inverse proportion to ‘voltage’ (see voltage in the denominator), so that a device with lower driving voltage becomes much advantageous from the aspect of electric power consume.

Power Efficiency (lm/W)=(π×luminous efficiency)/(current density×voltage) [Formula 1]

Thus, the OLEDs of Example 1 and Example 2 employing metal complexes according to the present invention provides the results of having excellent power efficiency of at least two times in low current density to high current density as compared to OLED device by means of conventional material. Particularly, in the device of Example 2 wherein light emitting layer and electron transport layer are used together, the increase of efficiency of about three times was confirmed.

Table 1 below shows light emitting properties of complexes developed according to the present invention. In terms of performances, excellent properties can be confirmed as compared to conventional materials.

TABLE 1
OLED properties of each compound
(luminescent material B: (NPy)₂Ir(acac))

| | | Driving | Luminous | | |
|---|---|---|---|---|---|
| | | voltage | efficiency | Power efficiency | |
| | | (V) | (cd/A) | (lm/W) | |
| Luminescent | | @ 88 | @ 2,000 | @ 2,000 | @ 10,000 |
| material A | ETL | MA/cm² | cd/m² | cd/m² | cd/m² |
| BAlq | Alq | 10.0 | 11.3 | 4.7 | 2.8 |
| Zn-DPBT | Alq | 8.5 | 16.3 | 8.3 | 4.5 |
| (Syn. Ex. 2) | | | | | |
| Zn-DPBT | Zn- | 5.2 | 16.1 | 14.3 | 8.1 |
| (Syn. Ex. 2) | DPBT | | | | |
| Zn-DMBT | Alq | 6.5 | 13.5 | 9.0 | 6.1 |
| (Syn. Ex. 4) | | | | | |
| Zn-3-DPDB | Alq | 8.1 | 17.5 | 9.5 | 5.0 |
| (Syn. Ex. 6) | | | | | |
| Zn-4-DPDB | Alq | 6.9 | 14.8 | 9.5 | 4.7 |
| (Syn. Ex. 8) | | | | | |

As can be seen from Table 1, when the metal complex of precursor having dimmer structure according to the present invention, in general, EL performance exhibit remarkable enhancement.

### INDUSTRIAL APPLICABILITY

The metal complex luminescent material comprised of precursor according to the present invention and metal significantly exhibits performances that lowers driving voltage in OLED device and remarkably raises luminous efficiency, so that the material is suitable for material for next generation and is expected to significantly contribute to manufacture of OLED in large scale.

1. A precursor of metallic complex for electroluminescent material represented by Chemical Formula 1. 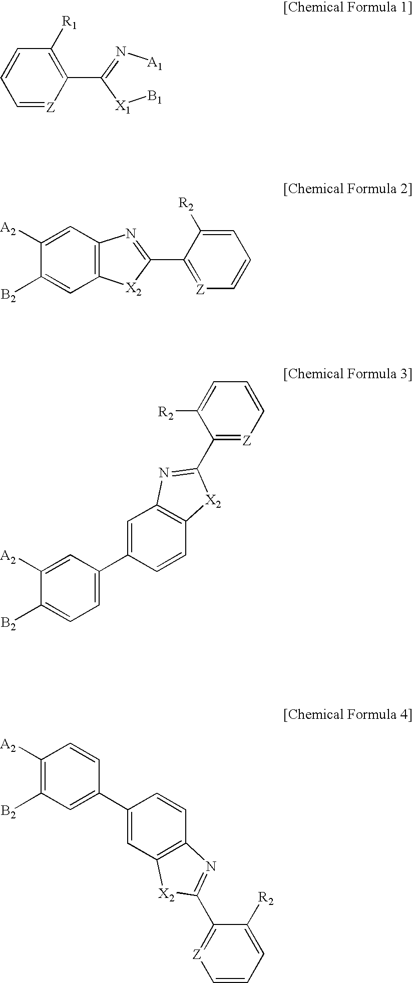
wherein, a compound represented by Chemical Formula 1 forms A₁-A₂/B₁—B₂ or A₁-B₂/B₁-A₂ single bond with each substituent selected from the groups represented by one of Chemical Formulas 2 to 4; if Z is carbon (C), each X₁ and X₂ independently represents O, S, Se or N-Ph, and each R₁ and R₂ independently represents NH₂, OH or SH; if Z is nitrogen (N), each X₁ and X₂ independently represents NH or PH, and each R₁ and R₂ is hydrogen.

2. A precursor of metallic complex for electroluminescent material according to claim 1, which is represented by one of Chemical Formulas 5 to 8: 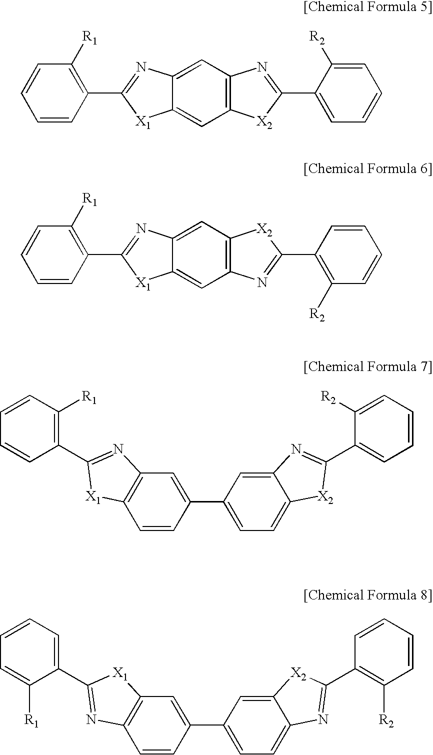
wherein, each X₁ and X₂ independently represents O, S, Se or N-Ph, and each R₁ and R₂ independently represents NH₂, OH or SH.

3. A precursor of metallic complex for electroluminescent material according to claim 1, which is represented by one of Chemical Formulas 9 to 12: 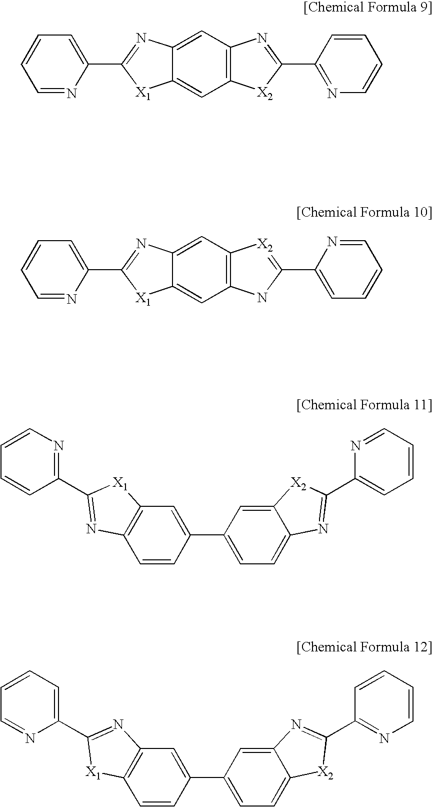
wherein, each X₁ and X₂ independently represents NH or PH.

4. A precursor of metallic complex for electroluminescent material according to claim 2, which is selected from the compounds represented one of following Chemical Formulas:

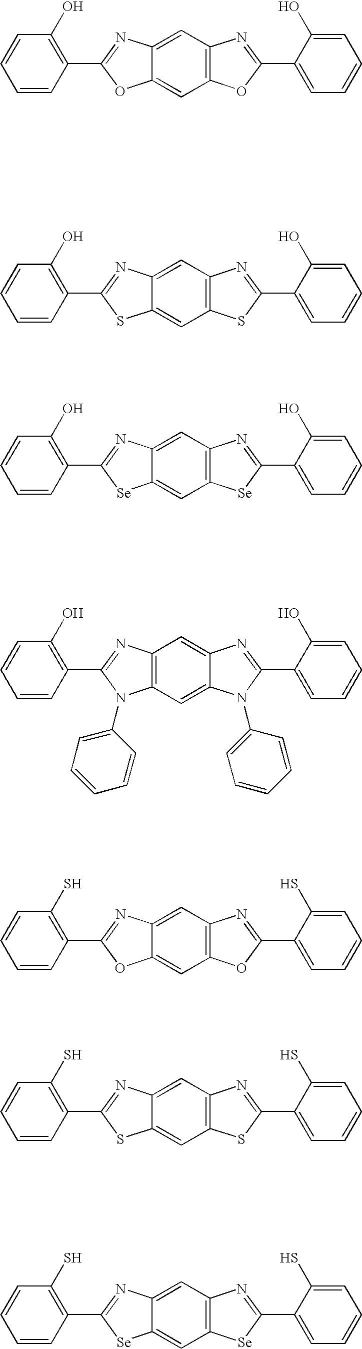

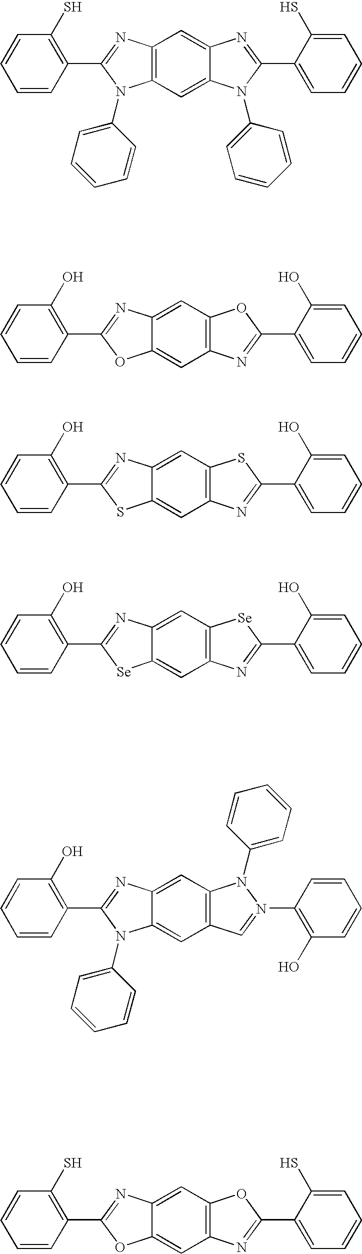

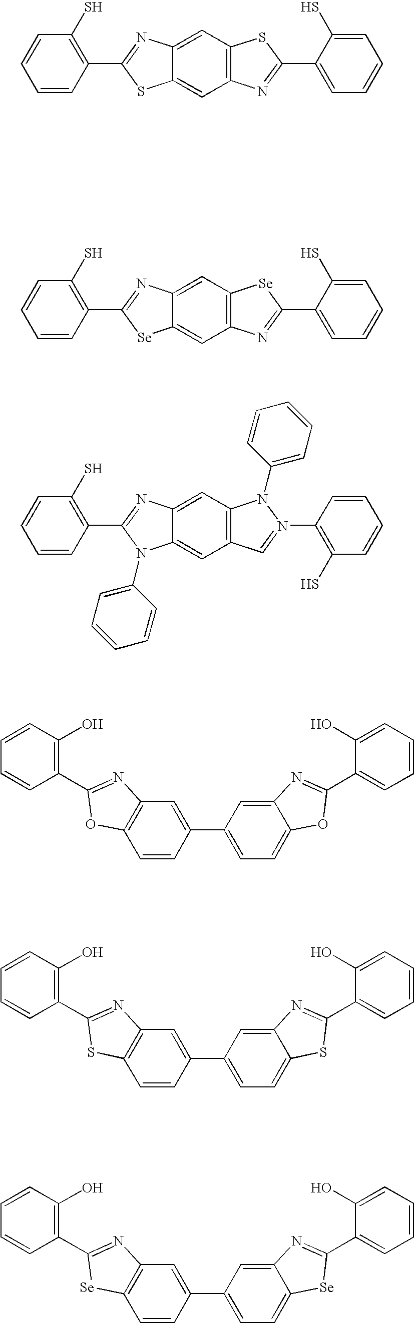

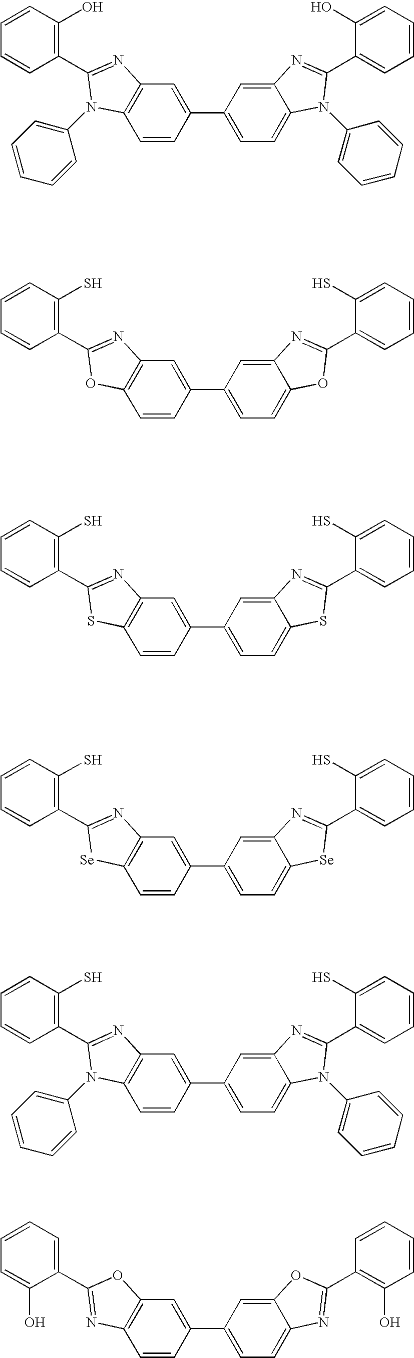

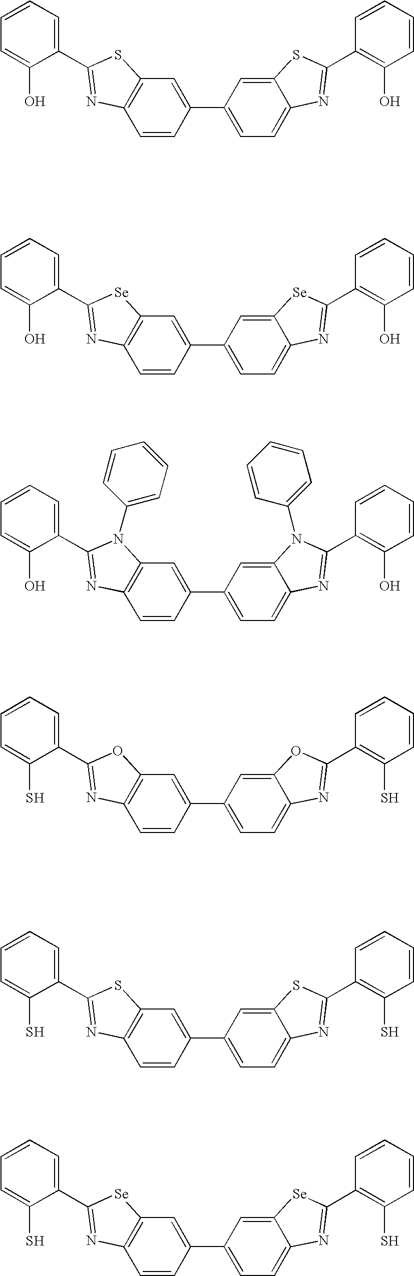

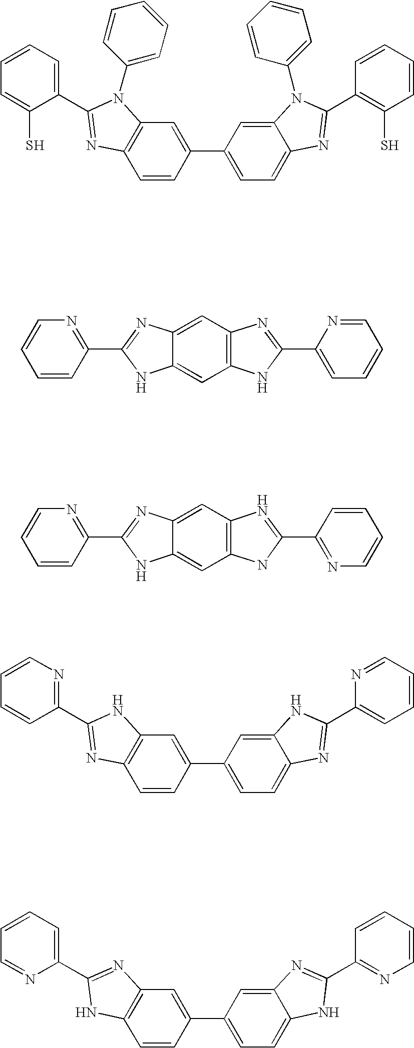

5. A luminescent material of metallic complex comprised of a precursor according to claim 1 and metal.

6. A luminescent material of metallic complex comprised of a precursor according to claim 5, characterized in that the metal is selected from the group consisting of Be, Zn, Mg and Al.

7. An electroluminescent device characterized in that a luminescent material of metallic complex is contained between the anode and the cathode.

8. A precursor of metallic complex for electroluminescent material according to claim 3, which is selected from the compounds represented one of following Chemical Formulas:

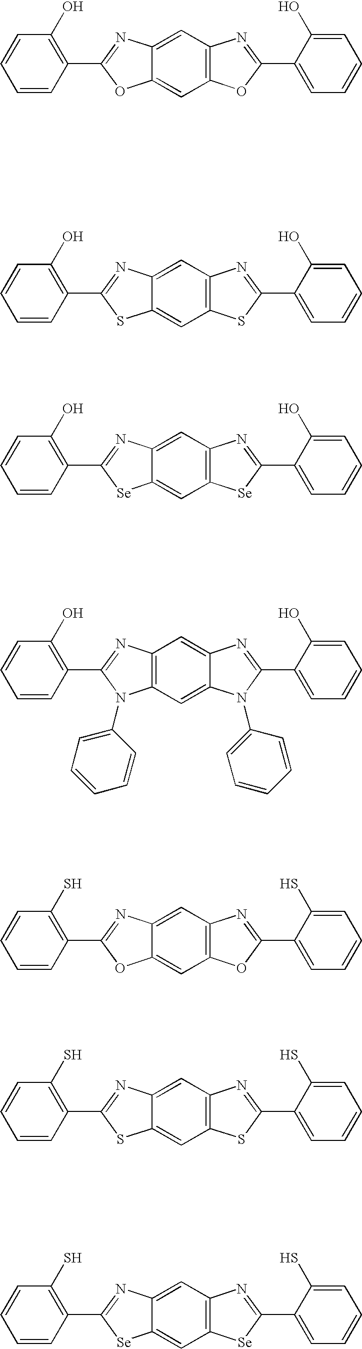

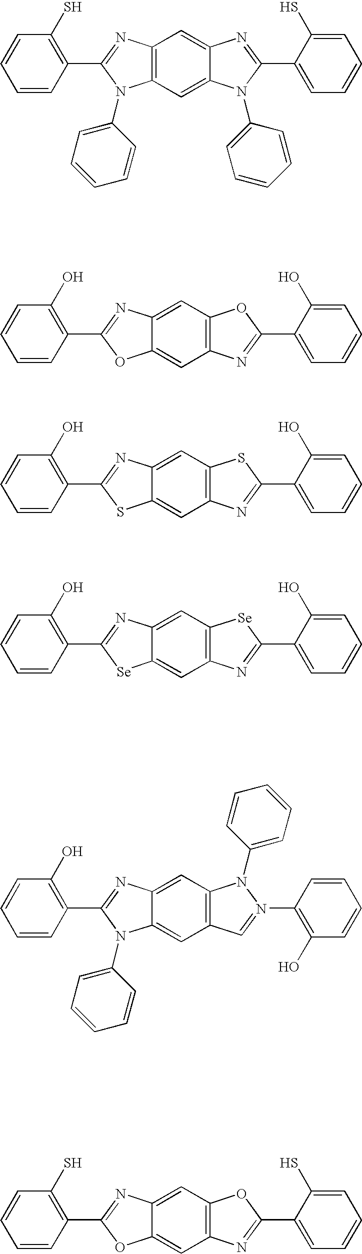

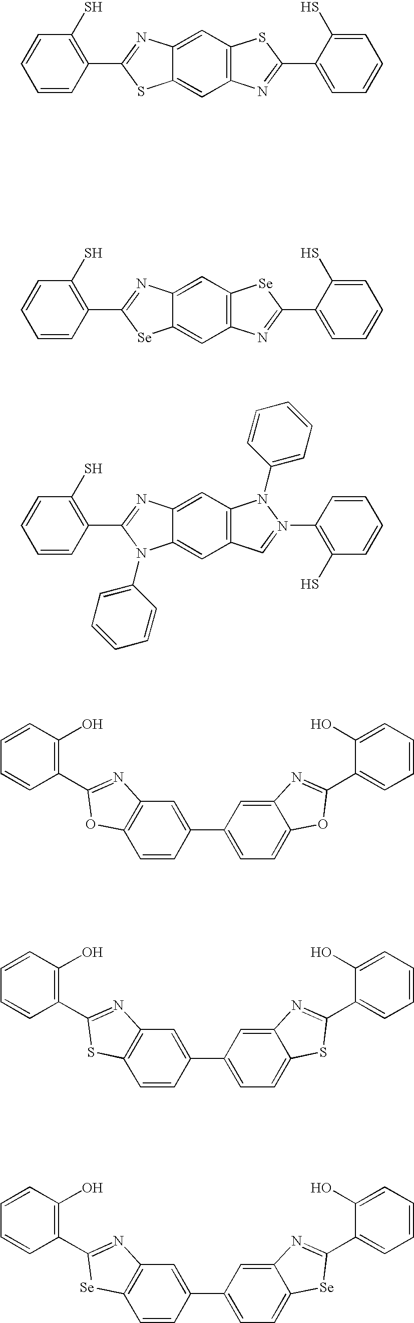

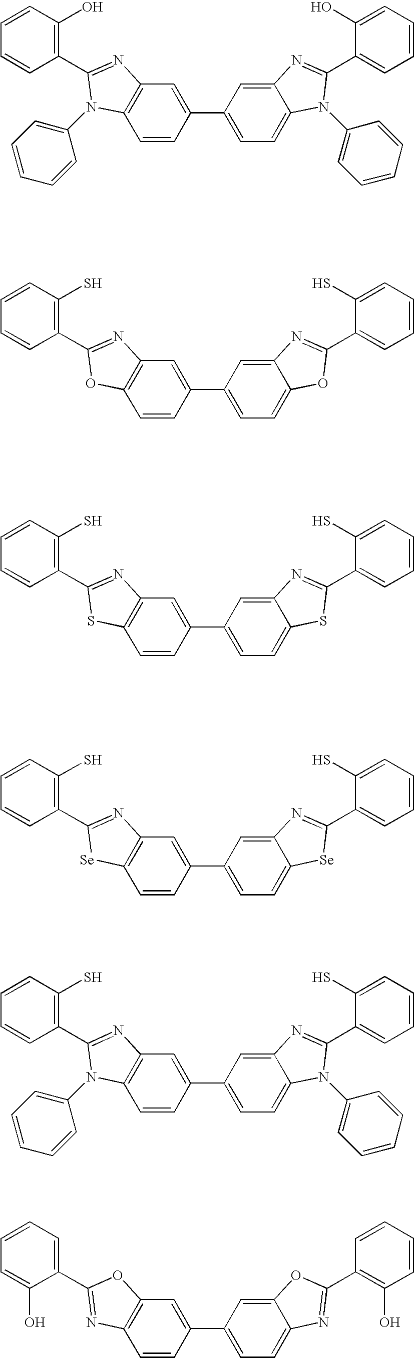

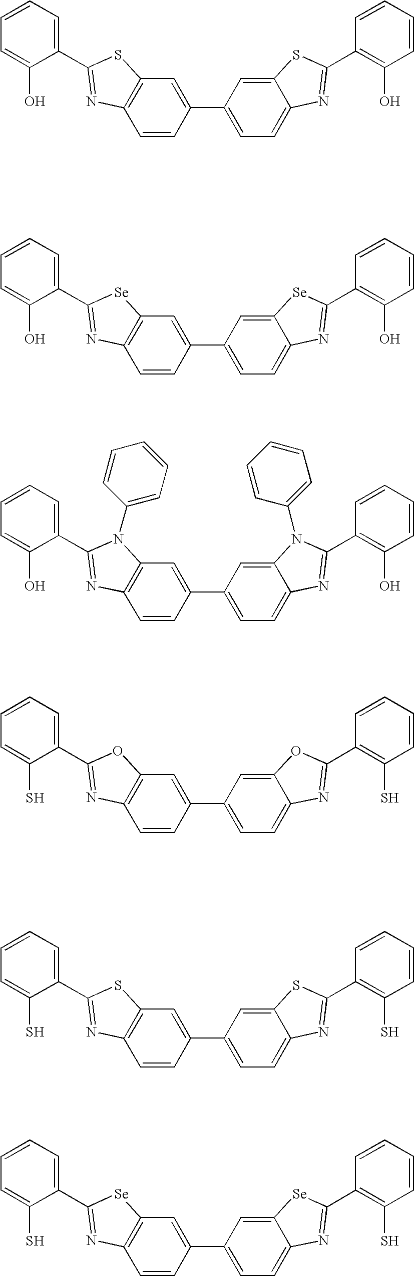

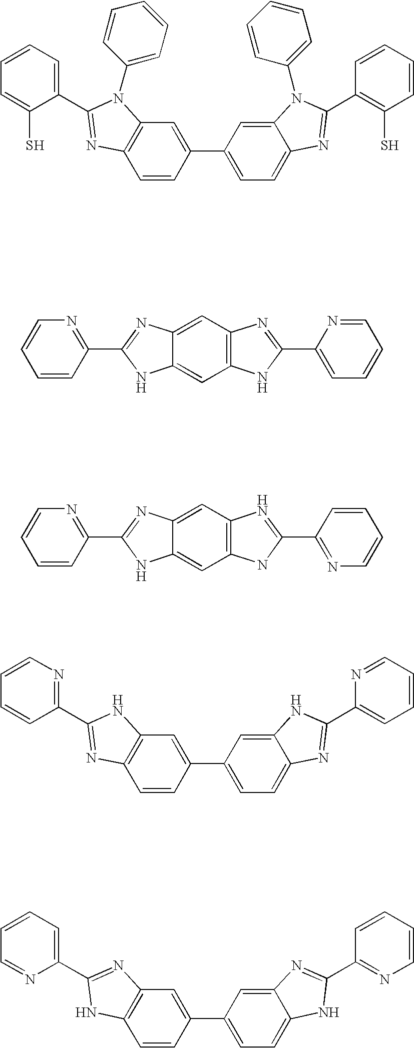

9. A luminescent material of metallic complex comprised of a precursor according to claim 2 and metal.

10. A luminescent material of metallic complex comprised of a precursor according to claim 3 and metal.

11. A luminescent material of metallic complex comprised of a precursor according to claim 4 and metal.
